(19) United States
(12) Patent Application Publication
(10) Pub. No.: US 2025/0057857 A1
(43) Pub. Date: Feb. 20, 2025

(54) AQUEOUS PARENTERAL PHARMACEUTICAL FORMULATIONS CONTAINING 4-((2-HYDROXY-3-METHOXYBENZYL)AMINO) BENZENESULFONAMIDE DERIVATIVES

(71) Applicant: Veralox Therapeutics Inc., Frederick, MD (US)

(72) Inventors: David MALONEY, Point of Rocks, MD (US); Matthew BOXER, New Market, MD (US); Richard WINNIKE, Raleigh, NC (US)

(21) Appl. No.: 18/723,045

(22) PCT Filed: Dec. 15, 2022
(86) PCT No.: PCT/US2022/081645

§ 371 Date: Jun. 21, 2024

Related U.S. Application Data
(60) Provisional application No. 63/292,759, filed on Dec. 22, 2021.

(51) Int. Cl. A61K 31/635; A61K 9/00; A61K 47/40

(52) U.S. Cl. CPC A61K 31/635; A61K 9/0019; A61K 47/40

(57) ABSTRACT

Provided are aqueous pharmaceutical formulations comprising 4-((2-hydroxy-3-methoxybenzyl)amino)-benzenesulfonamide 12-LOX inhibitors with improved solubility. Also provides are aqueous parenteral pharmaceutical formulations comprising selective 12-LOX inhibitors with 2-hydroxyalkyl-β-cyclodextrins.

### CROSS-REFERENCE TO RELATED APPLICATIONS

This patent application is an international patent application which claims priority to U.S. Provisional Application No. 63/292,759 filed on Dec. 22, 2021, the disclosure of which is incorporated herein in its entirety.

### BACKGROUND OF THE INVENTION

Lipoxygenases are a class of non-heme iron-containing enzymes which regio- and stereospecifically oxidize polyunsaturated fatty acid substrates such as arachidonic acid (AA) and linoleic acid (LA). (Solomon, et al. Chem. Biol. 1997, 4, 795-808; Brash, J. Biol. Chem. 1999, 274, 23679-23682.) The position at which these cis, cis-1,4-pentadiene substrates are oxidized correspond to the requisite lipoxygenase, with the three major human lipoxygenases: 5-LOX, 12-LOX, and 15-LOX-1, oxidizing the C-5, C-12 and C-15 positions respectively. Lipoxygenases are involved in the first committed step in a cascade of metabolic pathways and the products of these enzymes (eicosanoids) are precursors of hormones such as leukotrienes and lipoxins, which mediate a wide array of cellular functions. (Serhan, et al. Chem. Rev. 2011, 111, 5922-5943). Consequently, the lipoxygenase enzymes and their bioactive metabolites (e.g., hydroxyeicosatetraenoic acid (HETE) and leukotriene A4) have been implicated in a variety of inflammatory diseases and cancers.

12-LOX exists as three isozymes, platelet-type, leukocyte, and epidermal, but leukocyte 12-LOX is found in rat, mouse, pig and cow, but not in humans. (Yamamoto Biochim. Biophys. Acta. 1992, 1128, 117-131; Funk et al. FEBS Lett. 1997, 402, 162-166).

12-LOX has been demonstrated to play a role in a number of conditions and/or diseases, such as skin diseases and platelet hemostasis, transplantation/xenotransplantation, cancer (including but not limited to prostate cancer, colorectal cancer, breast cancer and lung cancer), type 1 and type 2 diabetes, diabetic kidney disease (diabetic nephropathy), diabetic nerve disease, cardiovascular disease (including but not limited to myocardial infarction, congestive heart failure, heart failure, and stroke), thrombosis, heparin induced thrombocytopenia (HIT), Alzheimer's disease, non-alcoholic steatohepatitis, insulin resistance, and inflammation.

A 4-((2-hydroxy-3-methoxybenzyl)amino)-benzenesulfonamide-based scaffold was identified and used for medicinal chemistry optimization and biological characterization as described in Luci, D K. et al. J. Med. Chem. 2014, 57, 495-506, and U.S. Pat. Nos. 10,266,488 and 10,752,581. This optimization produced several highly potent, selective 12-LOX inhibitors, e.g., N-(benzo[d]thiazol-2-yl)-4-((2-hydroxy-3-methoxybenzyl)amino)benzenesulfonamide (Compound 1), N-(benzo[d]oxazol-2-yl)-4-((2-hydroxy-3-methoxybenzyl)amino)benzenesulfonamide (Compound 2), and 4-((2-hydroxy-3-methoxybenzyl)amino)-N-(3-isopropylphenyl)benzenesulfonamide (Compound 3), TFA.

A challenge for the therapeutic use of 4-((2-hydroxy-3-methoxybenzyl)amino)-benzenesulfonamide and related 12-LOX inhibitors are poor aqueous solubility (<5 μM). There exists a need for aqueous pharmaceutical formulations of 4-((2-hydroxy-3-methoxybenzyl)amino)-benzenesulfonamide 12-LOX inhibitors.

Relatively insoluble compounds, i.e., solubility in water of less than 200 g/mL may show promising pharmaceutical activity, but their development as pharmaceuticals, particularly in parenteral dosage form, present a challenge. Non-aqueous solvents have long been used in parenteral pharmaceutical formulations to dissolve water-insoluble drugs. However, these organic solvents, regarded as chemically and biologically inert, may show pharmacological and toxicological effects. These methods are often inadequate for solubilizing enough of a quantity of a drug for a parenteral formulation. In addition, only therapeutic compounds dissolved in body fluids can permeate biological barriers and reach their site of action on a protein or cell surface or within certain intracellular structures.

### BRIEF SUMMARY OF THE INVENTION

The present application provides aqueous pharmaceutical formulations for parenteral administration, including intravenous administration, comprising 4-((2-hydroxy-3-methoxybenzyl)amino)-benzenesulfonamide 12-LOX inhibitors. Preferred are methods of administering these formulations as therapeutic agents in the treatment or prevention of 12-LOX mediated diseases and disorders by intravenous infusion.

The present disclosure in one embodiment provides an aqueous pharmaceutical formulation comprising:

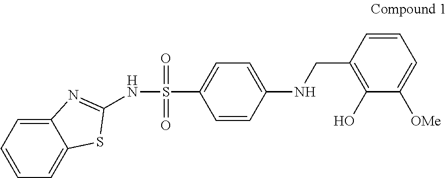 [SMILES: COc1cccc(CNc2ccc(S(=O)(=O)Nc3nc4ccccc4s3)cc2)c1O]

or a pharmaceutically acceptable salt thereof; and
2-hydroxypropyl-β-cyclodextrin (HP-β-CD) in an amount of between about 1% and 50% w/v,
wherein the formulation has a pH of between about 6.0 and 10.

In another embodiment, the present disclosure provides an aqueous pharmaceutical formulation comprising:

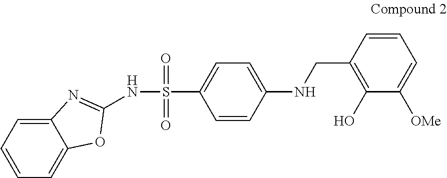 [SMILES: COc1cccc(CNc2ccc(S(=O)(=O)Nc3nc4ccccc4o3)cc2)c1O]

or a pharmaceutically acceptable salt thereof, or diastereomers thereof, and
2-hydroxypropyl-β-cyclodextrin (HP-β-CD) in an amount of between about 1% and 50% w/v,
wherein the formulation has a pH of between about 6.0 and 10.

In another embodiment, the disclosure provides an aqueous pharmaceutical formulation comprising:

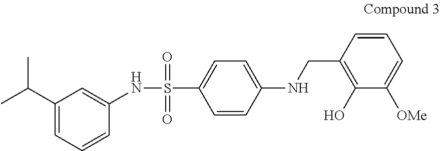 [SMILES: COc1cccc(CNc2ccc(S(=O)(=O)Nc3cccc(C(C)C)c3)cc2)c1O]

or a pharmaceutically acceptable salt thereof, or diastereomers thereof; and
2-hydroxypropyl-β-cyclodextrin (HP-β-CD) in an amount of between about 1% and 50% w/v,
wherein the formulation has a pH of between about 6.0 and 10.

In one embodiment, the aqueous pharmaceutical formulation comprises 2-hydroxypropyl-β-cyclodextrin (HP-β-CD) is in an amount between about 5%-15%, 10%-20%, 15%-25%, 25%-40%, 35%-45%, or 45%-50% w/v.

In a specific embodiment, the 2-hydroxypropyl-β-cyclodextrin (HP-β-CD) is in an amount of about 10%, 15%, 20%, 25%, 30%, 35%, 40%, 45%, or 50% w/v.

In another embodiment, the compound of Formula (I) is in an amount between about 1 mg/mL to about 100 mg/mL.

In a further embodiment, the compound of Formula (I) is in an amount between about 10-90 mg/mL, 50-100 mg/mL, 20-50 mg/mL, or 35-95 mg/mL.

In one embodiment, the aqueous pharmaceutical formulation wherein the composition has a pH in the range of 8.0 and 9.5, preferably between 8.4 to 8.7.

In a further embodiment, the formulation further comprises a pharmaceutically acceptable carrier, additive, excipient, preservative, solvent, buffer, or a mixture thereof.

In a specific embodiment, the formulation is suitable for parenteral administration, including subcutaneous injection, intravenous injection, intramuscular injection, intraperitoneal injection, infusion techniques, or a combination thereof. Intravenous formulations are preferred.

In another embodiment, the aqueous pharmaceutical formulation is administered by infusion. The infusion can comprise infusing between about 1 and 1,000 mg of compound 1 over 30 minutes to 24 hours, preferably for about 1, 2, 3, 4, or 5 hours.

In one embodiment, the amount of the compound 1 infused is about 10 to 1,000 mg.

In a further embodiment, the infusion is repeated over between about 1 and 14 days.

In one embodiment, the administration is by a bolus.

In other embodiment, the aqueous pharmaceutical formulation is used as a therapeutic agent in the treatment or prevention of 12-LOX mediated diseases and disorders in human and animal subjects, preferably mammals, more preferably humans.

In specific embodiments, wherein the 12-LOX mediated disease and/or disorder is selected from the group consisting of: type 1 diabetes, type 2 diabetes, diabetic kidney disease, diabetic nerve disease, cardiovascular disease, Alzheimer's disease, Non-Alcoholic steatohepatitis, platelet hemostasis, skin diseases, heparin induced thrombocytopenia, thrombosis, and cancer.

The present disclosure further proves method of making an aqueous pharmaceutical formulation by
(a) dissolving hydroxypropyl-β-cyclodextrin (HP-β-CD) in water to obtain a clear HP-β-CD solution;
(b) adding sodium hydroxide to the HP-β-CD solution while stirring to obtain a basic HP-β-CD solution of between about 9.5 and 12;
(c) adding the compound of Formula (I) slowly under agitation;
(d) optionally adding further sodium hydroxide while stirring;
(e) stirring for at least 10 minutes, optionally followed by at least 10 minutes of sonication at least three times until the compound of Formula (I) is dissolved; and (f) adding hydrochloric acid while stirring to result in a solution with a pH in a range from 8.4 to 8.7
(g) q.s. to final volume with water.

### BRIEF DESCRIPTION OF THE DRAWINGS

FIG. 1 depicts a solubility test for a 4-((2-hydroxy-3-methoxybenzyl)amino)-benzenesulfonamide 12-LOX inhibitor (Compound 1, VLX-1005) in a variety of solvents.

FIG. 2 depicts a solubility test for a 4-((2-hydroxy-3-methoxybenzyl)amino)-benzenesulfonamide 12-LOX inhibitor (Compound 1, VLX-1005) in a variety of solubilizers.

FIG. 3 is a flow-chart depicting an embodiment of the process of making a formulation for a 4-((2-hydroxy-3-methoxybenzyl)amino)-benzenesulfonamide 12-LOX inhibitor (Compound 1, VLX-1005) in a (2-Hydroxypropyl)-β-cyclodextrin (HP-β-CD).

### DETAILED DESCRIPTION OF THE INVENTION

The disclosure is not limited to the particular embodiments described below, as variations of the particular embodiments may be made and still fall within the scope of the appended claims. The terminology employed is for the purpose of describing particular embodiments and is not intended to be limiting.

### 4-((2-HYDROXY-3-METHOXYBENZYL)AMINO)-BENZENESULFONAMIDE DERIVATIVE 12-LIPOOXYGENASE INHIBITORS

Described herein are aqueous parenteral formulations comprising 12-LOX inhibitors. The term “parenteral,” as used herein, includes, but is not limited to, subcutaneous injections, intravenous, intramuscular, intraperitoneal injections, or infusion techniques, including bolus.

The aqueous parenteral pharmaceutical formulations described herein comprise a selective 12-lipoxygenase (12-LOX) inhibitor. In one embodiment, the selective 12-LOX inhibitor is a 4-((2-hydroxy-3-methoxybenzyl)amino)-benzenesulfonamide derivative. In a specific embodiment, the 4-((2-hydroxy-3-methoxybenzyl)amino)-benzenesulfonamide derivative is a compound of Formula (I):

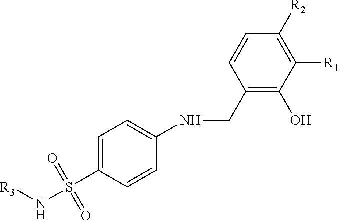

wherein R₁ is methoxy; R₂ is H; and R₃ is selected from the group consisting of 2-benzothiazole, 4-methyl-2-benzothiazole, 6-fluoro-2-benzothiazole, 6-methoxy-2-benzothiazole, 2-benzoxazole, 2-benzimidazole, 2-thiophene, 4-methyl-2-thiazole, 5-methyl-2-thiazole, 4,5-methyl-2-thiazole, 4-phenyl-2-thiazole, 3-quinoline, 8-isoquinoline, phenyl, 1,4-biphenyl, 1-naphthalene, 2-naphthalene, 3-piperazine-phenyl, 4-piperidine-phenyl, 3-piperidine-phenyl, 2-pyridine, 3-pyridine, 4-piperazine-3-pyridine, 3-tert-butyl-phenyl, 3-morpholine-phenyl, 4N-boc-piperidine-3-phenyl, and 3-isopropyl-phenyl.

In another embodiment, the aqueous parenteral pharmaceutical formulation comprises Compound 1:

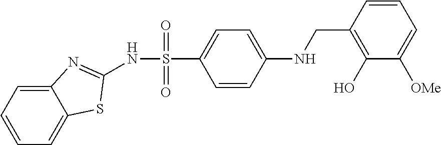 [SMILES: COc1cccc(CNc2ccc(S(=O)(=O)Nc3nc4ccccc4s3)cc2)c1O]

or a pharmaceutically acceptable salt thereof.

In another embodiment, the aqueous parenteral pharmaceutical formulation comprises Compound 2:

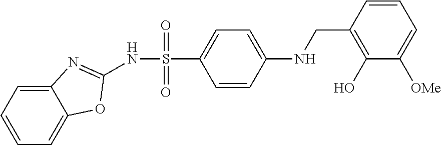 [SMILES: COc1cccc(CNc2ccc(S(=O)(=O)Nc3nc4ccccc4o3)cc2)c1O]

or a pharmaceutically acceptable salt thereof.

In a further embodiment, the aqueous parenteral pharmaceutical formulation comprises Compound 3:

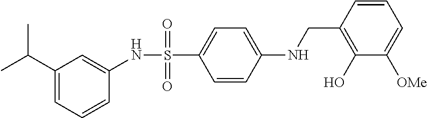 [SMILES: COc1cccc(CNc2ccc(S(=O)(=O)Nc3cccc(C(C)C)c3)cc2)c1O]

or a pharmaceutically acceptable salt thereof.

The aqueous parenteral pharmaceutical formulations described herein comprise the compounds or salt of Formula (I), or a combination thereof.

The present disclosure is further directed to an aqueous parenteral pharmaceutical formulation comprising an aqueous pharmaceutically acceptable carrier and at least one selective 12-LOX inhibitor compound described herein. In a preferred embodiment, the carrier is water, preferably sterile water.

Compounds 1, 2, and 3 were previously identified as selective 12-LOX inhibitors in e.g., U.S. Pat. No. 10,266,488:

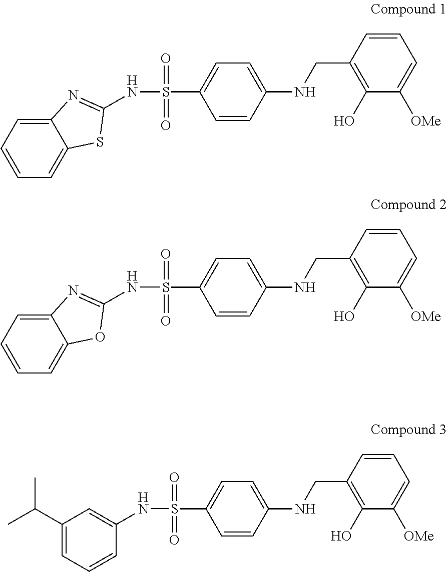 [SMILES: COc1cccc(CNc2ccc(S(=O)(=O)Nc3nc4ccccc4s3)cc2)c1O ; COc1cccc(CNc2ccc(S(=O)(=O)Nc3nc4ccccc4o3)cc2)c1O ; COc1cccc(CNc2ccc(S(=O)(=O)Nc3cccc(C(C)C)c3)cc2)c1O]

These compounds have low solubility in water. For example, Compound 1 (also known as ML355) was tested for solubility in various solvents, solubilizers and buffers for potential use in aqueous and non-aqueous solvents for intravenous administration. The results are described in detail in the Examples. Compound 1 was highly soluble in polar solvents, such as dimethyl sulfoxide (DMSO), dimethylacetamide (DMAC), and N-methyl-2-pyrrolidone (NMP), but insoluble in non-polar solvents, such as hexane. Compound 1 was also poorly soluble in aqueous pH buffers with the highest solubility observed at pH 10 (0.4-0.6 μg/mL) and no detectable solubility at pH 1.0-9.0. Solubility of Compound 1 in conventional solubilizers, such as Cremophor® RH40 (polyethylene glycol-40 hydrogenated castor oil), Solutol® HS 15 (polyoxyethylated 12-hydroxystearic acid), and Tween® 80 (polyoxyethylene (20) sorbitan monooleate), was also low.

Surprisingly, use of specific cyclodextrins improved solubility compared to use of other solubilizers. Specifically, the solubility of Compound 1 was nearly two times greater in HP-β-CD (2-hydroxypropyl-β-cyclodextrin) compared with Captisol (SBE-β-CD; sulfobutylether-β-cyclodextrin) at the same concentration (20%). HP-β-CD is a cyclic oligosaccharide containing seven D-(+)-glucopyranose units. Captisol is a polyanionic β-cyclodextrin with a sodium sulfonate salt separated from the lipophilic cavity by a butyl ester spacer group. Use of other 2-hdyroxyalkyl β-cyclodextrins can also be incorporated, specifically 2-hydroxybutyl-β-cyclodextrin, 2-hydroxyethyl-β-cyclodextrin, 2-O-(2-hydroxybutyl)-beta-cyclodextrin (2-HB-β-CD), and 6-O-(2-hydroxybutyl)-beta-cyclodextrin (6-HB-β-CD).

The expression “therapeutically effective amount” refers broadly to an amount of a compound disclosed herein, that is effective for preventing, ameliorating, treating or delaying the onset of a disease or condition. The expression “prophylactically effective amount” refers to an amount of a compound disclosed herein, that is effective for inhibiting the onset or progression of a disorder.

Formulations. It is known to one skilled in the art that certain 2-hydroxyalkyl-β-cyclodextrins could function as pharmaceutically acceptable solubilizers for various compounds in an aqueous parenteral pharmaceutical formulation; however, surprisingly it was found that only when the 12-LOX compounds were introduced to the HP-β-CD solution at a basic pH was there significant solubility enhancement. In one embodiment, the aqueous pharmaceutical formulations comprise a 2-hydroxyalkylated β-cyclodextrin including without limitation 2-hydroxypropyl-β-cyclodextrin (HP-β-CD), 2-hydroxyethyl-β-cyclodextrin (HE-β-CD) and 2-hydroxybutyl-β-cyclodextrin (HB-β-CD). Preferred is use of the pharmaceutically acceptable solubilizer HP-β-CD.

The pharmaceutically acceptable 2-hydroxyalkylated β-cyclodextrin such as HP-β-CD may be present in any suitable amount within the aqueous parenteral pharmaceutical formulations described herein. The pharmaceutically acceptable 2-hydroxyalkylated 3-cyclodextrin such as HP-β-CD may be present in amount of between about 5% and 50% w/v. For example, the pharmaceutically acceptable solubilizer, optionally HP-β-CD, may be in an amount of about 10%, 15%, 20%, 25%, 30%, 35%, 40%, 45%, or 50% w/v. In specific embodiments, the HP-β-CD may be present in amount from about 10% to about 40%, from about 20% to about 40%, from about 30% to about 40%, from about 10% to about 30%, or from about 10% to about 20% of the total formulation. In one embodiment, the 2-hydroxyalkylated 3-cyclodextrin, optionally, HP-β-CD may present in amount of about 25% of the total formulation w/v.

The 2-hydroxyalkylated β-cyclodextrin, optionally, HP-β-CD, may be present in the aqueous parenteral pharmaceutical formulation in amount of at least 100 mg/mL, at least 150 mg/mL, at least 200 mg/mL, at least 225 mg/mL, at least 250 mg/mL, at least 275 mg/mL, at least 280 mg/mL, or at least 300 mg/mL. The HP-β-CD may be present in the pharmaceutical formulation in amount from about 100 mg/mL to about 300 mg/mL, from about 200 mg/mL to about 300 mg/mL, from about 220 mg/mL to about 260 mg/mL, from about 200 mg/mL to about 275 mg/mL, or from about 210 mg/mL to about 255 mg/mL of the total formulation.

The aqueous parenteral pharmaceutical formulations described herein may comprise a compound described herein at an amount between about 1.0 mg/mL and 20 mg/mL, 2-hydroxyalkylated β-cyclodextrin, optionally, HP-β-CD, in an amount between about 1% and 30% w/v, a base (e.g., sodium hydroxide) and/or an acid (e.g., HCl). For example, the aqueous parenteral pharmaceutical formulations described herein may comprise a compound described herein at an amount between about 1.5 and 20 mg/mL and 2-hydroxyalkylated β-cyclodextrin, optionally, HP-β-CD, sodium hydroxide, and hydrochloric acid. The pharmaceutical formulations described herein may comprise a compound described herein at an amount of 15 mg/mL 2-hydroxyalkylated β-cyclodextrin, optionally, HP-β-CD, sodium hydroxide, and hydrochloric acid.

The pH of the aqueous parenteral pharmaceutical formulations described herein may be between about pH 6.0 and pH 10.0. The pH of the pharmaceutical formulations described herein may be at about pH 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In a preferred embodiment, the pH of the aqueous parenteral pharmaceutical formulations described herein may be between about pH 7.4 and pH 9.0. In an embodiment, the pH of the aqueous parenteral pharmaceutical formulations described herein may be at about pH 8.0, 8.1, 8.2, 8.3, 8.4, 8.5, 8.6, 8.7, 8.8, 8.9, or 9.0. The pH of the pharmaceutical formulations described herein may be at about pH 8.4, 8.5, 8.6, or 8.7. In another preferred embodiment, the pH of the aqueous parenteral pharmaceutical formulations described herein may be between a pH of about 8.4 and 8.7.

The solubility of the compounds in the aqueous parenteral pharmaceutical formulation may increase with increasing pH. In one embodiment, the aqueous pharmaceutical formulation comprises 20 mg/mL Compound 1 at pH 8.0. In another embodiment, aqueous parenteral pharmaceutical formulation comprises 40 mg/mL Compound 1 at pH 9.5. pH can be adjusted to provide a formulation comprising a desired dose.

In one embodiment, the aqueous parenteral pharmaceutical formulations described herein comprises a compound described herein and a pharmaceutically acceptable solubilizer, namely HP-β-CD. The pharmaceutical formulations described herein comprise Compound 1, Compound 2, Compound 3, or mixtures thereof.

In another embodiment, the pharmaceutically acceptable solubilizer is 2-hydroxyethyl-β-cyclodextrin (HE-β-CD) or 2-hydroxybutyl-β-cyclodextrin (HB-β-CD). Preferred is use of the pharmaceutically acceptable solubilizer HP-β-CD.

The aqueous parenteral pharmaceutical formulation may further contain other acceptable liquid carriers in an amount that does not alter the aqueous nature of the formulation, including, vegetable oils such as peanut oil, cotton seed oil, sesame oil, as well as organic solvents, PEG, propylene glycol, glycerol, and surfactants.

In one embodiment, the aqueous parenteral pharmaceutical formulation comprises at least 50% water, preferably 70% or more of water.

The compounds described herein may be present in any suitable amount within the aqueous parenteral pharmaceutical formulations described herein. Those of skill in the art can readily determine suitable concentrations of compound to include in the aqueous parenteral pharmaceutical formulations depending on various factors including dosage and route of administration. The aqueous parenteral pharmaceutical formulations useful in the present invention can contain a quantity of a compound described herein in an amount effective to treat or prevent the condition, disorder or disease of the subject being treated.

The compounds described herein, e.g., compounds of Formula (I), optionally Compound 1, Compound 2, Compound 3, or mixtures thereof, may be present in the aqueous parenteral pharmaceutical formulation in an amount of at least 0.1 mg/mL, at least 0.5 mg/mL, at least 1 mg/mL, at least 1.5 mg/mL, at least 2 mg/mL, at least 5 mg/mL, at least 10 mg/mL, at least 15 mg/mL, at least 20 mg/mL, at least 23 mg/mL, at least 25 mg/mL, at least 26 mg/mL, at least 28 mg/mL, or at least 30 mg/mL.

The compounds described herein may be present in the aqueous parenteral pharmaceutical formulation in an amount from about 0.1 mg/mL to about 50 mg/mL, from about 0.5 mg/mL to about 100 mg/mL, from about 1 mg/mL to about 100 mg/mL, from about 15 mg/mL to about 60 mg/mL, from about 20 mg/mL to about 40 mg/mL, from about 50 mg/mL to about 100 mg/mL, from about 10 mg/mL to about 50 mg/mL, from about 15 mg/mL to about 30 mg/mL, from about 25 mg/mL to about 75 mg/mL, or from about 50 mg/mL to about 100 mg/mL.

The compounds described herein may be present in the aqueous parenteral pharmaceutical formulation in an amount from about 0.1 mg/mL to about 50 mg/mL, from about 0.1 mg/mL to about 25 mg/mL, from about 0.1 mg/mL to about 20 mg/mL, from about 0.1 mg/mL to about 15 mg/mL, from about 0.1 mg/mL to about 10 mg/mL, from about 0.1 mg/mL to about 5 mg/mL, from about 0.1 mg/mL to about 3 mg/mL, from about 0.1 mg/mL to about 2 mg/mL, from about 0.1 mg/mL to about 1.5 mg/mL, or from about 0.1 mg/mL to about 1 mg/mL. In some embodiments, the compound of Formula (I) is present in the pharmaceutical formulation in amount from about 1 mg/mL to about 20 mg/mL or from about 1.5 mg/mL to about 30 mg/mL.

The compounds described herein may be present in the aqueous parenteral pharmaceutical formulation in an amount of about 0.1 mg/mL, about 0.5 mg/mL, about 1 mg/mL, about 1.5 mg/mL, about 2 mg/mL, about 5 mg/mL, about 10 mg/mL, about 15 mg/mL, about 20 mg/mL, about 25 mg/mL, about 50 mg/mL or about 100 mg/mL.

The inventors surprisingly discovered that the solubility of the Compounds 1, 2, and 3, is greatly enhanced in a pH dependent manner in the presence of cyclodextrin. For example, at pH 8.0 the solubility of the compounds is about 20 mg/mL with the 25% cyclodextrin and at pH 9.8 it doubles to about 40 mg/mL.

In addition to the compounds disclosed herein, the aqueous parenteral pharmaceutical formulations may further comprise at least one of any suitable auxiliaries including, but not limited to, diluents, crystal inhibitors, tonicifiers, water structure forming agents or disruptors, polymers, ion pairing agents, stabilizers, buffers, salts, lipophilic solvents, preservatives, adjuvants or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Adejare, Ed., 23rd Edition, Academic Press. (2020); Handbook of Pharmaceutical Excipients, 9^{th} Edition, Pharmaceutical Press (2020)). Pharmaceutically acceptable carriers can be routinely selected that are suitable for the mode of administration, solubility and/or stability of the compound.

Pharmaceutical excipients and additives useful in the aqueous parenteral pharmaceutical formulations described herein can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, and casein. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, and aspartame.

Carbohydrate excipients suitable for use in the aqueous parenteral pharmaceutical formulations described herein include but are not limited to monosaccharides such as dextrose, fructose, maltose, galactose, glucose, D-mannose, and sorbose; disaccharides, such as lactose, sucrose, trehalose, and cellobiose; polysaccharides, such as raffinose, melezitose, maltodextrins, dextrans, and starches; and alditols, such as mannitol, xylitol, maltitol, lactitol, xylitol, sorbitol (glucitol), and myoinositol.

The pharmaceutical formulations comprising the compounds described herein can also a pH adjusting agent. The pH adjusting agent may be a base, optionally sodium hydroxide.

The present disclosure provides stable aqueous parenteral pharmaceutical formulations as well as preserved solutions and formulations containing a preservative, as well as multi-use preserved formulations suitable for pharmaceutical or veterinary use, comprising at least one compound disclosed herein in a pharmaceutically acceptable formulation. Pharmaceutical formulations in accordance with the present disclosure may optionally comprise at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% phenol, 0.0005-1.0% alkylparaben(s).

Other excipients, e.g., isotonicity agents, buffers, antioxidants, preservative enhancers, can be optionally added. When used, the amount of tonicity modifier used most often ranges from 0.1 to 1% (w/v). Non-limiting examples of suitable tonicity modifiers include sodium chloride, glycerin, boric acid, calcium chloride, dextrose, and potassium chloride. The formulations can include a local anesthetic to reduce the potential of pain during injection. A physiologically tolerated buffer can be added to provide improved pH control if necessary. The pharmaceutical formulations can cover a wide range of pHs, such as from about pH 4 to about pH 10, specifically, a range from about pH 5 to about pH 9, and more specifically, a range of about 7.0 to about 9.0. In one aspect, the formulations described herein have pH between about 8.4 and about 8.7.

The pharmaceutical formulations described herein can also include additional therapeutic agents such as argatroban, but not limited other therapeutic agents or combinations thereof.

An additional therapeutic agent may be an additional 12-LOX inhibitor. A 12-LOX inhibitor can be an organic compound, an inorganic compound, a biological compound (e.g., proteins or fragments thereof, antibodies or fragments thereof, nucleic acids, nucleic acid analogs, saccharides, or peptides), or any combination thereof. A 12-LOX inhibitor can also be synthetic or naturally occurring. Selective 12-LOX inhibitors are described in U.S. Pat. Nos. 10,266,488 and 10,752,581.

The mixing ratios of the 12-LOX inhibitors may be optimized to provide maximum therapeutic effects.

Additional agents include but not limited to anti-thrombotic agents such as argatroban, fondaparinux, lepirudin, bivalirudin, danaparoid and drotrecogin alfa; antidiabetic agents such as exenatide, albiglutide, pramlintide, semaglutide, lixisenatide, and dulaglutide. Combinations with direct-acting oral coagulants is also contemplated, including but not limited to apixaban, dabigatran, rivaroxaban, and edoxaban.

Aqueous parenteral pharmaceutical formulations suitable for parenteral administration include aqueous sterile injection solutions which may comprise anti-oxidants, bacteriostats and solutes that render the formulation isotonic with the blood of the intended recipient. The formulations may be presented in unit-dose or multi-dose containers, sealed ampules and vials, and may be stored in a freeze-dried (lyophilized) condition requiring only the addition of the sterile liquid carrier, e.g., water for injections, saline, dextrose in water (D5W), lactated Ringer's solution, immediately prior to use. Extemporaneous injection solutions and suspensions may be prepared from sterile powders, granules and tablets of the kind previously described.

For parenteral administration, sterile solutions and suspensions are desired. Isotonic preparations which generally contain suitable preservatives are employed when intravenous, administration is desired. The aqueous pharmaceutical formulations may be administered parenterally via injection of a pharmaceutical formulation comprising a compound dissolved in an inert liquid carrier, such as sterile water or other pharmaceutically acceptable diluents. The term “parenteral,” as used herein, includes, but is not limited to, subcutaneous injections, intravenous, intramuscular, intraperitoneal injections, or infusion techniques, including bolus.

Methods of preparing aqueous pharmaceutical formulations with a certain amount of active ingredients are known, or will be apparent in light of this disclosure, to those skilled in the art. Methods of preparing said pharmaceutical formulations can incorporate other suitable pharmaceutical excipients and their formulations as described in REMINGTON'S PHARMACEUTICAL SCIENCES, above.

Methods of preparing the aqueous pharmaceutical preparations described herein are manufactured in a manner that is known, including conventional mixing, dissolving, or lyophilizing processes. Thus, aqueous pharmaceutical preparations can be obtained by combining the active compounds with solid excipients, optionally grinding the resulting mixture and processing the mixture of granules, after adding suitable auxiliaries, if desired or necessary.

In one embodiment, the cyclodextrin is dissolved in water while being subjected to a first pH adjustment with a base to bring the pH up to about 9.5-12.5 to enhance dissolution of the compound. This followed by addition of the compound. Once the compound is completely in solution, the pH is brought down to a physiological range with acid to the physiological pH of e.g., between about 8.4-8.7.

Administration and Dosage. One of ordinary skill in the art will appreciate that administration of pharmaceutically effective amounts of the aqueous parenteral pharmaceutical formulations described herein to a patient in need thereof, can be determined empirically, or by standards currently recognized in the medical arts. It will be understood that, when administered to a human patient, the total daily usage of the agents of the compositions described herein will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Routes of administration and dosages of effective amounts of the aqueous parenteral pharmaceutical formulations comprising the compounds are also disclosed. The preferred route of administration is intravenous. The pharmaceutical formulations described herein may also be administered by infusion. The pharmaceutical formulations described herein may also be administered by a bolus dosage, optionally combined with administration by infusion. The compounds described herein can be administered in combination with other pharmaceutical agents in a variety of protocols for effective treatment of disease.

The aqueous parenteral pharmaceutical formulations may be administered in a single daily dose, or the total daily dosage may be administered in divided doses of two, three, or four times daily. Doses maybe administered for one week, one month, or over the course of several months, 3, 6, 9 or 12 months, or intervals known in the art and determined to be clinically relevant. For example, the pharmaceutical formulations described herein may be administered for 7 days, 14 days, 21 days, 28 days, or 35 days. The daily dosage of the formulations may be varied over a wide range from about 0.0001 to about 1,000 mg per patient, per day, more particularly from about 200 to about 700, preferably about 600 mg/day. The range may more particularly be from about 1 mg/kg to 100 mg/kg of body weight per day, about 10-50 mg/kg, 20 to 60 mg/kg, preferably 10-20 mg/kg per day for adults (at about 60 kg), or equivalent doses as determine by a practitioner, to achieve a serum concentration that is clinically relevant.

Specifically, the aqueous parenteral pharmaceutical formulations described herein may be administered at least once a day over the course of several weeks, several months, or several years. In one embodiment, the pharmaceutical formulations are administered at least once a day over several weeks to several months. In another embodiment, the pharmaceutical formulations are administered once a day over at least one year.

The aqueous parenteral pharmaceutical formulations may be administered following use of a topical anesthetic including but not limited to lidocaine, prilocaine, or combinations thereof. Ice or ethyl chloride spray can also be used prior to administration

It can be sometimes desirable to deliver the compounds described herein to the subject over prolonged periods of time, for periods of one week to one year from a single administration. Certain medical devices may be employed to provide a continuous intermittent or on demand dosing of a patient. The devices may be a pump of diffusion apparatus, or other device containing a reservoir of drug and optionally diagnostic or monitoring components to regulate the delivery of the drug. Various slow-release, depot or implant dosage forms can be utilized.

In one embodiment, the aqueous parenteral pharmaceutical formulations described herein may be administered by infusion. The infusion may comprise infusing between about 1 and 600 mg of the 12-LOX inhibitor in the aqueous pharmaceutical formulation continuously, e.g., over 24 hours. In another embodiment, the infusion is administered over 30 minutes to 23 hours. The infusion may be for about 1, 2, 3, 4, 5, 6, 7, 8, 9, 10, 11, 12, 13, 14, 15, 16, 17, 18, 19, 20, 21, 22, or 23 hours. The amount of the 12-LOX inhibitor infused may be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mg.

In one embodiment, the infusion may be continuous or repeated over intervals. In one embodiment, the intervals are between about 1 day to 1 year, or between about 1 day to 6 months, or 1 day to 1 month. In another embodiment, the infusion may be repeated over between about 1 and 14 days, optionally between about 1 and 7 days. The infusion may be repeated over 1, 2, 3, 4, 5, 6, 7, 8, 9, 10, 11, 12, 13, or 14 days. The infusion may be repeated over between about 1 and 7 days, 3 and 8 days, 5 and 14 days, 9 and 14 days, or 7 and 14 days. For example, the pharmaceutical formulations described herein may be infused continuously or over a period of 1 to 23 hours, including all intervals in between, at a rate of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180, 190 mg, 200 mg, 210 mg, 220 mg, 230 mg, 240 mg, 250 mg, 260 mg, 270 mg over 1-5 hours, 280 mg, 290 mg, 300 mg, 310 mg, 320 mg, 330 mg, 340 mg, 350 mg, 360 mg, 370 mg, 380 mg, 390 mg, 400 mg, 510 mg, 520 mg, 530 mg, 540 mg, 550 mg, 560 mg, 570 mg, 580 mg, 590 mg, or 600 mg. In a specific embodiment, infusion occurs over 1 to 12 hours, 1 to 6 hours, or 1 to 4 hours.

The aqueous parenteral pharmaceutical formulations described herein can be administered to any animal that can experience the beneficial effects of the compounds of the invention. Preferred is administration to humans.

The dosage administered will be dependent upon the age, health, and weight of the recipient, kind of concurrent treatment, if any, frequency of treatment, and the nature of the effect desired.

### Therapeutic and Prophylactic Methods 12-LOX Mediated Diseases/Disorders

The disclosure further provides a method for treating or preventing a 12-LOX mediated disease or disorder comprising administering to a mammal a therapeutically or prophylactically effective amount of an aqueous parenteral pharmaceutical formulation described herein.

The 12-LOX mediated disease or disorder to be treated or prevented is typically a disease or disorder wherein the production of 12-hydroxyeicosatetraenoic acid (12-HETE) is implicated in the development or progression of the disease or disorder. 12-LOX mediated diseases and disorders includes those where 12-LOX is a direct mediator of the diseases and disorders, as well as those where the inhibition of 12-LOX results in therapeutic value in the treatment or prevention of the diseases and disorders.

A method for treating or preventing a 12-LOX mediated disease or disorder may comprise administering to a mammal a therapeutically or prophylactically effective amount of any of compounds of Formula (I), or a salt thereof in the aqueous parenteral pharmaceutical formulation. In one embodiment, the compound in the formulation is Compound 1.

In one embodiment, the 12-LOX is human 12-LOX.

The 12-LOX mediated disease or disorder may be selected from the group consisting of type 1 diabetes, type 2 diabetes, diabetic kidney disease, diabetic nerve disease, cardiovascular disease, Alzheimer's disease, Non-Alcoholic steatohepatitis, platelet hemostasis, skin diseases, heparin induced thrombocytopenia, thrombosis, and cancer.

The cancer may be selected from the group consisting of prostate cancer, colorectal cancer, breast cancer, and lung cancer. In another embodiment, the cancer is a hematologic cancer.

The cardiovascular disease may be selected from the group consisting of congestive heart failure, myocardial infarction and stroke.

A method of treating or preventing type 1 and/or type 2 diabetes may comprise administering to a mammal a therapeutically or prophylactically effective amount of any of compounds described herein, e.g., compounds of Formula (I), Compound 1, Compound 2, Compound 3, or mixtures thereof, or a salt thereof in the aqueous parenteral pharmaceutical formulation.

A method of treating or preventing thrombosis may comprise administering to a mammal thereof an aqueous parenteral formulation comprising a therapeutically or prophylactically effective amount of any of the compounds described herein, a salt thereof.

A method for reducing PAR4-AP induced platelet aggregation may comprise administering to a mammal thereof an aqueous parenteral formulation comprising a therapeutically or prophylactically effective amount of any of compounds described herein, e.g., compounds of Formula (I), Compound 1, Compound 2, Compound 3, or a mixture thereof, a salt thereof.

A method for reducing PAR4-AP induced calcium mobilization may comprise administering to a mammal thereof an aqueous parenteral formulation comprising a therapeutically or prophylactically effective amount of any of compounds described herein, a salt thereof.

12-LOX inhibitors may be used in transplantation/xenotransplantation methods, for example, where islets are treated ex vivo to improve survival prior to transplant.

Provided is a method for treating or preventing a disease or disorder in which a FcγRIIa-mediated pathway is involved comprising administering an aqueous parenteral formulation described herein. Such a disease or disorder can be an immune-mediated thrombocytopenia and thrombosis disorder. Examples of immune-mediated thrombocytopenia and thrombosis disorders include, but are not limited to, heparin-induced thrombocytopenia (HIT); anti-phospholipid syndrome; sepsis syndrome; thrombosis associated with therapeutic or diagnostic monoclonal antibodies; and thrombotic thrombocytopenic purpura. In some aspects, disclosed herein are methods for inhibiting platelet activation and preventing or treating thrombosis.

A method of inhibiting or decreasing platelet activation may comprise contacting a platelet with an aqueous parenteral formulation comprising an effective amount of a compound described herein. In some embodiments, the platelet activation is immune-mediated. In some embodiments, the immune-mediated platelet activation is resulting from the activation of a FcγRIIa receptor.

Methods for treating or preventing a thrombotic event, myocardial infarction, or stroke may comprise administering to a subject in need thereof an aqueous parenteral formulation comprising an effective amount of a compound described herein are also disclosed. Immune-mediated platelet activation can lead to thrombi formation, which can clot arteries and result in stroke, myocardial infarction, organ infarction, limb gangrene, or other serious complications.

The subject can be one who exhibits one or more risk factors for an immune-mediated thrombocytopenia and thrombosis disorder. The subject may be receiving or had recently received heparin therapy. The subject may be undergoing orthopedic surgery. It is known in the art that orthopedic surgery patients are at higher risk for developing HIT than are patients who receive heparin for other medical reasons.

A method for treating or preventing a 12-LOX mediated disease and/or disorder may administer the aqueous pharmaceutical formulation comprising Compound 1, Compound 2, Compound 3, or a combination thereof to a subject in need thereof. The subject in need thereof may be a mammal. In a preferred embodiment, the subject in need thereof is a human.

The 12-LOX mediated disease and/or disorder may be type 1 diabetes, type 2 diabetes, diabetic kidney disease, diabetic nerve disease, cardiovascular disease, Alzheimer's disease, Non-Alcoholic steatohepatitis, platelet hemostasis, skin diseases, heparin induced thrombocytopenia, thrombosis, lupus, or cancer.

A method for treating a patient suffering from immune-mediated thrombocytopenia may comprise administering the aqueous parenteral pharmaceutical formulation comprising Compound 1, Compound 2, Compound 3, or a combination thereof, to a subject in need thereof.

A method for treating a patient suffering from a thrombosis disorder comprising administering a parenteral pharmaceutical formulation comprising Compound 1, Compound 2, Compound 3, or a combination thereof to a subject in need thereof.

The aqueous parenteral pharmaceutical formulation may be administered by infusion. The infusion may comprise infusing between about 1 and 600 mg of the 12-LOX inhibitor in the aqueous pharmaceutical formulation over 30 minutes to 24 hours. The infusion may be for about 1, 2, 3, 4, or 5 hours. The amount of the 12-LOX inhibitor infused may be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg. The infusion may be repeated over between about 1 and 14 days. The infusion may be repeated over 14 days or longer. The infusion may be repeated over 28 days or longer.

The aqueous parenteral pharmaceutical formulations described herein may be administered by a bolus. The amount of the 12-LOX inhibitor in a formulation administered in a bolus may be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg.

### Methods of Formulation

The compounds described herein, e.g., compounds of Formula (I), for example Compound 1, Compound 2, and Compound 3, show poor solubility in water. The inventors surprisingly found that the combination of 2-hydroxypropyl-β-cyclodextrin (HP-β-CD) and a high pH, e.g., between about 8.4 and 8.7, resulted in increased solubility of the compounds. Related 2-hydroxyalkyl-β-cyclodextrins such as HE-P-CD and HB-β-CD may also be useful.

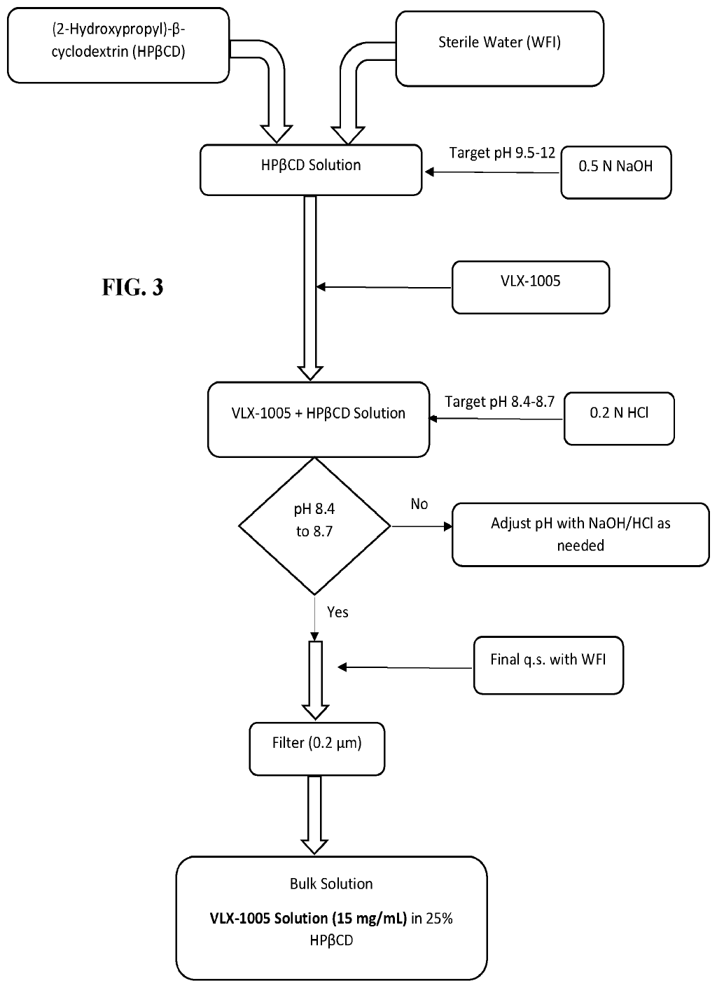

A solution formulated for intravenous infusion may be made by the process shown in FIG. 3. In an embodiment, the cyclodextrin is dissolved in water along with a base to bring the pH up to about 9.5-12.5, followed by addition of the compound. Once the compound is completely in solution, the pH is lowered to a physiological range with an acid to a pH of e.g., between about 8.4-8.7. In a specific embodiment, HP-β-CD, or a related 2-hydroxyalkyl-β-cyclodextrin, or combinations thereof, is dissolved in water with agitation, e.g., vigorous stirring, until a clear solution is obtained. Dilute sodium hydroxide is then slowly added to the e.g., HP-ββ-CD solution while continuously stirring. The resulting basic pH of between about 9.5 to 12.5 facilitates the dissolution of the compounds described herein. A compound described above, e.g., Compound 1, is then slowly added to the cyclodextrin under vigorous stirring. Stirring is continued for e.g., an additional 10 min followed by 10 min of sonication. Alternate stirring and sonication cycles are repeated at least three (3) times until the active ingredient is dissolved. For example, 3 cycles of 10 minutes of stirring and 10 minutes of sonication. Under continuous stirring, dilute hydrochloric acid is slowly added to a target pH range of 8.4 to 8.7. Water may be added to achieve the target concentration of the compound per mL, e.g., 30 mg/mL, 15 mg/mL, 1.5 mg/mL. The solution may be filtered through a 0.22 μm filter and stored at 2-8° C. Prior to administration, the solution is sterile filtered under aseptic processing into syringes, vials, or ampules of appropriate size. In one embodiment, dissolution is performed at room temperature, i.e., about 20-25° C. or lower.

Although preferred embodiments have been depicted and described in detail herein, it will be apparent to those skilled in the relevant art that various modifications, additions, substitutions, can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, can be performed without altering the spirit or scope of the invention, and such modifications and variations are encompassed within the scope of the invention as defined in the claims which follow. The following examples do not in any way limit the invention.

### EXAMPLES

### Example 1: Solubility Study of Compound 1

Solubility analysis of Compound 1 (N-(benzo[d]thiazol-2-yl)-4-((2-hydroxy-3-methoxybenzyl)amino)benzenesulfonamide) was performed in a total of 25 media including organic solvent, co-solvent, solubilizer and buffer, for 2 hours at room temperature.

For the solubility tests, Compound 1 was in anhydrous form with rod shaped crystalline, high crystallinity and high melting point at 231° C. The particle size was 50-250 μm.

Results showed that the compound was highly soluble in DMSO, DMAC and NMP at neutral pH (7.4) with a solubility of more than 100 mg/mL at 0.5 h. Compound 1 was also soluble in PEG400 with more than 10 mg/mL at 2 h. In other solvents, the solubility was low than 1 mg/mL. The rank order from high to low was DMSO>DMAC=NMP>PG>EtOH>Glycerin>n-Hexane.

For solubilizers, HP-β-CD exhibited good solubilization at neutral pH (7.4) when the content was 20% or greater. The highest solubility was 0.57 mg/mL in 40% HP-β-CD at 2 h. The solubility increased >8 fold with the increase of HP-β-CD from 10% to 40%. Solubilization in Captisol was weaker than HP-β-CD at the same content. 5% Solutol showed better solubilization than other surfactants, while the solubility was still low (˜0.1 mg/mL at 2 hours).

Solubility in pH buffers was very low, the main peak of the compound was not detected by HPLC at pH 1-9 (<0.15 μg/mL) and solubility in pH 10 was 0.4-0.6 μg/mL. The relative ratio of impurity at 1.63 min increased in pH 9-10 buffers at 2 h, which might be due to the higher solubility of the impurity in the initial sample or degradation of the compound in buffers.

### 1. Introduction

The purpose of this study is to measure the solubility of Compound 1 in various media for 2 hours at room temperature.

### 2. Material and Reagents

### 2.1. Material

The information of the compound is listed in Table 1.

TABLE 1
Information of the Starting Material

| Compound ID | MW | Lot # | Purity | Amount |
|---|---|---|---|---|
| 1 | 441.52 | 3 | >98% | ~410 mg |

### 2.2. Reagents

Information of reagents and excipients is listed in Table 2.

TABLE 2
Information of Reagents and Excipients

| Reagent | Company | Lot # |
|---|---|---|
| EtOH | Beijing Chemical Works | 20180622 |
| n-Hexane | Beijing Chemical Works | 20180604 |
| PEG400 | Wako pure chemical Industries. Ltd | WEF3466 |
| PG | Sinopharm Chemical Reagent Co., Ltd | 20160318 |
| Glycerin | Tianjin Guangfu Fine Chemical | 20181021 |
| | Research Institute | |
| DMAC | Beijing Jingqiu Chemical Industry | 20161204 |
| | Product Co., Ltd. | |
| DMSO | Beijing Chemical Works | 20180710 |
| NMP | Tianjin Guangfu Fine Chemical | 20180704 |
| | Research Institute | |
| HP-β-CD | TCI | 4BRVM-QI |
| Captisol | Shanghai D.B. Biotechnology Co. Ltd | EP29 |
| Cremophor RH40 | BASF | 32177676K0 |
| Solutol | BASF | 14225516K0 |
| Tween 80 | Tianjin Guangfu Fine Chemical | 20160828 |
| | Research Institute | |
| Sodium citrate | Gu'an Xian Jingqiu Chemical | 20180313 |
| | Co., Ltd. | |
| Citric acid | Beijing Chemical Industry Co., Ltd. | 20181920 |
| NaHCO₃ | Beijing Jingqiu Chemical Industry | 20170625 |
| | Product Co., Ltd. | |
| NaOH | Beijing Chemical Works | 20170628 |
| ACN | Fisher Chemical | 184266 |
| Water | Watson Distilled Water | 20181110H |
| TFA | J&K | L4B0Q01 |

### 3. Analytical Method

### 3.1. Powder X-ray Diffraction (XRPD)

Solid samples were analyzed using X-ray diffractometer (Bruker D8 advance). The system was equipped with LynxEye detector. Samples were scanned from 3 to 40° 2θ, at a step size 0.02° 2θ. The tube voltage and current were 40 KV and 40 mA, respectively.

### 3.2. Thermogravimetric Analysis (TGA)

TGA was carried out on Discovery TGA 55. The sample was placed in an open tarred aluminum pan, automatically weighed, and inserted into the TGA furnace. The sample was heated at a rate of 10° C./min to the final temperature.

### 3.3. Differential Scanning Calorimetry (DSC)

DSC analysis was conducted on Discovery DSC 250. A sample in weight was placed into a DSC pinhole pan, and weight was accurately recorded. The sample was heated at a rate of 10° C./min to the final temperature.

### 3.4. Polarizing Microscopy Analysis (PLM)

PLM analyses were conducted on a Nikon Polarizing Microscope ECLIPSE LV100POL. Solid was placed on a glass slide and dispersed by cedar wood oil, then covered by coverslip.

### 3.5. High Performance Liquid Chromatography (HPLC)

HPLC method with acceptable resolution and main peak purity developed to determine solubility. The method is described in Table 3.

TABLE 3
HPLC Method

| | |
|---|---|
| Instrument | Agilent HPLC 1260 series |
| Flow rate | 1.0 mL/min |
| Injection Volume | 3 μL |
| Mobile phase | A: 0.1% TFA in water; B: 0.1% TFA in ACN |
| Column | Eclipse C18, 4.6*100 mm, 3.5 μm |
| Column temperature | 40° C. |
| Detector | DAD |
| Wavelength | 302 nm |
| Gradient (T/B %) | 0/45%, 6/60%, 8/100% |
| Run time | 8 minutes |
| Post time | 3 minutes |
| Diluent | ACN/water (1:1) + 5% DMSO |

### 4. Experimental Procedures

### 4.1. Solid State Characterization of the Starting Material

The starting material was characterized by XRPD, TGA, DSC and PLM.

### 4.2. Solubility Measurement in Solvents

About 5 mg of Compound 1 was weighed to sample vial and 0.5 mL of solvent was added (target concentration was 10 mg/mL) with 30-100 μL per time. The samples were shaken at room temperature. The samples were prepared in duplicate (n=2) per medium and filtered at 0.5 and 2 hours. The supernatant was collected and analyzed by HPLC.

### 4.3. Solubility Measurement in Solubilizers and Buffers

5-10 mg of Compound 1 was added to 0.5-1 mL each medium (target concentration was 10 mg/mL), vortexed briefly to create a suspension. The samples were shaken at room temperature. The samples were prepared in duplicate (n=2) per medium and filtered at 0.5 and 2 hours. The supernatant was collected and analyzed by HPLC.

### 5. Results

### 5.1. Solid State Characterization

The results are summarized in Table 4. The starting material was rod shaped crystalline with high crystallinity and high melting point. It was an anhydrous form. The particle size was ˜50-250 μm.

TABLE 4
Solid state characterization

| Item | Result |
|---|---|
| XRPD | Crystalline with high crystallinity |
| PLM | Rod shaped with particle size of 50-250 μm |
| TGA | No weight loss before decomposition |
| DSC (Onset-peak | An endothermic peak at 228.77-231.15° C., |
| Temp. ° C.) | and the enthalpy was 122 J/g. |

### 5.2. Solubility in Solvents

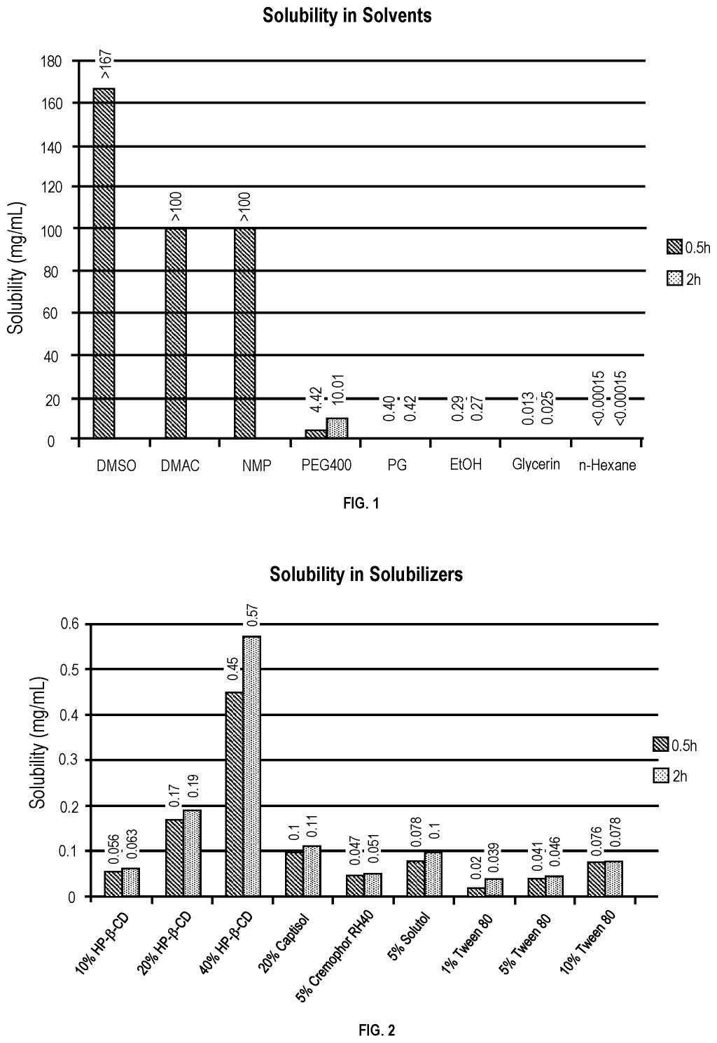

Solubility results are summarized in Table 5 and FIG. 1 The compound was highly soluble in polar solvent, including DMSO, DMAC and NMP, with the concentration of more than 100 mg/mL at 0.5 h. However, it was insoluble in hexane, a non-polar solvent, at less than 0.15 g/mL. The rank order from high to low was DMSO>DMAC=NMP>PG>EtOH>Glycerin>n-Hexane.

TABLE 5
Solubility Results in Solvents

| | Solubility at | Solubility at |
|---|---|---|
| Solvent | 0.5 h (mg/mL) | 2 h (mg/mL) |
| DMSO¹ | >167 | Not precipitated |
| DMAC¹ | >100 | Not precipitated |
| NMP¹ | >100 | Not precipitated |
| PEG400 | 4.42 | 10.00² |
| PG | 0.40 | 0.42 |
| EtOH | 0.29 | 0.27 |
| Glycerin | 0.013 | 0.025 |
| n-Hexane | <0.00015³ | <0.00015³ |

Note:
¹The solubilities in DMSO, DMAC and NMP were evaluated by visual observation
²The compound solution in PEG400 was clear at 2 h.
³LOQ = 0.00015 mg/mL.

### 5.3. Solubility in Solubilizers

HP-β-CD showed good solubilization effect when the content was 20% or greater. The highest solubility was 0.57 mg/mL in 40% HP-β-CD at 2 h. The solubility increased >8-fold with the increase of HP-β-CD from 10% to 40%. Solubilization of Captisol was weaker than HP-β-CD at the same content. HP-β-CD at a 40% w/v showed the best solubilization effects on Compound 1.

500 Solutol showed better solubilization effect than other surfactants, while the solubility was still low (˜0.1 mg/mL at 2 hours). The increase of Tween 80 from 1% to 10% could increase the solubility about 4-fold, but the highest solubility was only 0.078 mg/mL.

The results were summarized in Table 6 and FIG. 2.

TABLE 6
Solubility Results in Solubilizers in
aqueous phosphate buffer at pH 7.4

| | Solubility at | Solubility at |
|---|---|---|
| Solubilizer | 0.5 h (mg/mL) | 2 h (mg/mL) |
| 10% HP-β-CD | 0.056 | 0.063 |
| 20% HP-β-CD | 0.17 | 0.19 |
| 40% HP-β-CD | 0.45 | 0.57 |
| 20% Captisol | 0.10 | 0.11 |
| 5% Cremophor RH40 | 0.047 | 0.051 |
| 5% Solutol | 0.078 | 0.10 |
| 1% Tween 80 | 0.020 | 0.039 |
| 5% Tween 80 | 0.041 | 0.046 |
| 10% Tween 80 | 0.076 | 0.078 |

### 5.4. Solubility in Buffers.

Solubility in pH buffers was very low, the highest solubility was observed in pH 10 at 004-0.6 μg/mL, while the main peak of the compound cannot be detected by HPLC at pH 1-9. It was observed that the relative ratio of impurity at 1.63 min increased in pH 9-10 buffers at 2 h, which might be due to the higher solubility of the impurity in the initial sample or degradation of the compound in buffers. The results are summarized in Table 7.

TABLE 7
Solubility Results in Buffers Over a pH Range

| | Solubility (mg/mL) | | pH | |
|---|---|---|---|---|
| Buffer | 0.5 h | 2 h | 0.5 h | 2 h |
| Citrate buffer, pH 2.5 | N.D. | N.D. | 2.68 | 2.63 |
| Citrate buffer, pH 3.0 | N.D. | N.D. | 3.08 | 3.05 |
| Citrate buffer, pH 3.5 | N.D. | N.D. | 3.60 | 3.56 |
| Citrate buffer, pH 4.0 | N.D. | N.D. | 4.07 | 3.98 |
| Citrate buffer, pH 5.0 | N.D. | N.D. | 4.99 | 4.94 |
| Citrate buffer, pH 7.0 | N.D. | N.D. | 7.04 | 7.03 |
| Bicarbonate, pH 9.0 | N.D. | N.D. | 9.11 | 9.10 |
| Bicarbonate, pH 10.0 | 0.000397 | 0.0005764 | 10.02 | 10.08 |

### 6. Conclusion

Compound 1 was an anhydrous form with rod shaped crystalline, high crystallinity and high melting point at 231° C. The particles were block-shaped with sizes 50-250 μm.

Compound 1 was insoluble in the media containing water, its concentration in buffers was below detection and the highest was 0.4 μg/mL at pH 10. The solubility was >100 mg/mL in DMSO, DMAC, NMP; 4-10 mg/mL in PEG400. Solubilizers, HP-β-CD and surfactants provided some solubilization effect with HP-β-CD being most effective with a range of 0.05-0.5 mg/mL. The inventor surprisingly discovered that the combination of HP-β-CD and a pH between about 8.0 and 9.0, Compound 1 showed the best solubilization effects.

### Example 2: Solubility of Compound 1 in HP-β-CD at High pH

Compound forms a metastable supersaturated solution when formulated with cyclodextrins. To assess for increased solubility, Compound 1 was added to a basic cyclodextrin solution to effect dissolution. The resulting solution was then adjusted with hydrochloric acid into a physiologically compatible pH range and subjected to kinetic solubility, equilibrium solubility, and formulation solubility analyses. It was surprisingly discovered that the combination of HP-β-CD and a pH of greater than about 8.0, including between about 8.0 and 9.5, increased the solubility 50 to 100 times. Compound 1 showed the best solubilization effects.

Kinetic solubility. Initial kinetic solubility evaluations contained up to 40 mg/mL of Compound 1 in 25% HP-β-CD with high concentrations of sodium hydroxide (NaOH) relative to the Compound 1 (2.2:1 molar ratio, NaOH to Compound 1). The resulting intermediate pH values were approximately 12.5, which promoted rapid dissolution of Compound 1. However, the solutions are known to be chemically labile at higher pH values. The concentration of Compound 1 in subsequent formulation evaluations was lowered to 20 mg/mL and then reduced to 15 mg/mL.

Hydrochloric acid (1-C) was required to adjust the high intermediate pH Compound 1 formulations to an acceptable pH (˜8.5). The concentrated HCl produced a greater potential for Compound 1 precipitation when adjusting the formulation to a physiologically acceptable pH, due to the supersaturated state of the Compound 1 solutions. The resulting osmolality values were relatively high for the early prototype formulations due to the osmotic contributions of the concentrated pH modifiers. Various NaOH to Compound 1 ratios (ranging from 0.44:1 to 2.2:1 molar) were evaluated during the Compound 1/HP-β-CD solution development to determine the NaOH to Compound 1 ratio which would provide the best compromise between Compound 1 dissolution rate, osmolality, and stability.

Equilibrium solubility. Compound 1 equilibrium solubility was evaluated by adding excess Compound 1 to Compound 1 solution formulations. The equilibrium solubility samples were stored at room temperature and/or refrigerated (4-8° C.) conditions with continual mixing prior to analyzing. A NaOH to Compound 1 molar ratio of 1.6:1 was selected for the final 15 mg/mL Compound solution formulation as the best compromise between dissolution rate, hyper-osmolality, and chemical degradation. At the 1:6 NaOH:Compound 1 molar ratio, the drug is dissolved in a relatively short time frame, which minimizes high pH degradation and results in acceptable solution osmolality. The amount of Compound 1 did not increase significantly in the equilibrium solubility evaluations in which excess Compound 1 was added to Compound 1 solutions and mixed for extended times. The pH and equilibrium solubility data are listed in Table 8:

TABLE 8
Compound 1 Equilibrium Solubility Evaluations in 25% HP-β-CD

| | | | | | | | | | 4-8° C | |
|---|---|---|---|---|---|---|---|---|---|---|
| | | | | | | RT Assay | | | Assay | |
| | | | | | | (compound | | | (compound | |
| | compound | NaOH:compound | pH | | | 1 mg/mL) | | | 1 mg/mL) | |
| Batch | (mg/mL) | Molar Ratio | Initial | 24-hr | 48-hr | Initial | 24-hr | 48-hr | 24-hr | 48-hr |
| 2020/MA/0001- | 20 (10 + | 1.3:1 | 9.02 | 8.60 | 8.44 | 9.5^{B} | 9.3^{C} | 9.2 | N/A | N/A |
| 5B^{A} | 10) | | | | | | | | | |
| 2020/MA/0001- | 30 (20 + | 1.39:1 | 8.89 | N/A | N/A | 18.1^{B} | 17.9 | N/A | 18.2 | 18.8 |
| 16B | 10) | | | | | | | | | |
| 2020/MA/0001- | 30 (20 + | 1.6:1 | 8.56 | 9.12 | 8.91 | 20.1 | N/A | N/A | N/A | 18.3^{D} |
| 23B | 10) | | | | | | | | | |

^{A}Batches contained excess VLX-1005 which was added to parent batches. Batch 2020/MA/0001-5B was prepared by adding & mixing excess VLX-1005 (50.04 mg) to a 5-mL aliquot of batch 2020/MA/0001-5A.
^{B}low assay values attributed to HPLC diluent concentration; originally 60:40 ACN:Water was adjusted to 75:25 ACN:Water for 100% recovery
^{C}40 hour assay; 24-hour assay not obtained
^{D}Batch 2020/MA/0001-23B containing 10 mg/mL excess VLX-1005 (30 mg/mL total) was filtered and assayed after 7 days of mixing at 5° C.

The optimum molar ratio of sodium hydroxide to Compound 1 was determined using the solubility and osmolality data from the Compound 1/HP-β-CD development batches. The ratio of 1.6:1 sodium hydroxide to Compound 1 was selected for the final Compound 1/HP-β-CD formulation based on the comparative performance across the range of ratios evaluated. The resulting intermediate pH of the Compound 1/HP-β-CD solution was 11.4-11.6, prior to the final pH adjustment to a target pH of 8.5 with HC; (acceptable pH range 8.4-8.7).

Formulation stability of supersaturated solutions. Meta-stability of the supersaturated Compound 1 solution formulations were evaluated at various time points following storage at room temperature and refrigerated (4-8° C.) conditions. Batch sizes ranged from 5 to 700 mL. The VLX-1005 solution stability samples and placebo samples were prepared per the proposed clinical compounding procedures (Example 4, below). The stability data from various Compound 1 development batches are provided in Tables 9 and 10.

TABLE 9
Supersaturated Compound 1 in 25% HP-β-CD Solution Stability Evaluations

| | | | | | | | | | 4-8° C. | |
|---|---|---|---|---|---|---|---|---|---|---|
| | | NaOH:VLX- | | | | RT Assay | | | assay | |
| | | 1005 | pH | | | (mg/mL) | | | (mg/mL) | |
| Batch | mg/mL | Molar Ratio | Initial | 24-br | 48-hr | Initial | 24-hr | 48-hr | 24-hr | 48-hr |
| 2020/MA/0001- | 10 | 1.32:1 | 9.02 | N/A | 8.6 | 9.5 | 9.6 | 9.2 | N/A | N/A |
| SA | | | | | | | | | | |
| 2020/MA/0001- | 15 | 0.44:1 | 9.09 | 8.55 | 8.28 | 6.6A | 7.1A | 6.7A | N/A | N/A |
| 6A | | | | | | | | | | |
| 2020/MA/0001- | 20 | 0.66:1 | 9.52 | 9.12 | 8.91 | 13.3A | 13.3A | 12.7A | N/A | N/A |
| 6B | | | | | | | | | | |
| 2020/MA/0001- | 25 | 0.8:1 | 10.21 | 9.69 | 9.50 | 19.9 | 19.3 | 18.3 | N/A | N/A |
| 6C | | | | | | | | | | |
| 2020/MA/0001- | 25 | 0.8:1 | 9.72 | 9.86 | N/A | 18.9 | 18.2 | N/A | N/A | N/A |
| 6C^{B} | | | | | | | | | | |
| 2020/MA/0001- | 20 | 1.32:1 | 8.45 | N/A | 8.12 | 18.1 | 18.3 | 17.2 | 17.7 | 17.2 |
| 10 | | | | | | | | | | |
| 2020/MA/0001- | 20 | 1.32:1 | 8.80 | N/A | 8.77 | 19.2 | 19.2 | 18.7 | 18.9 | 18.4 |
| 11 | | | | | | | | | | |
| 2020/MA/0001- | 20 | 1.39:1 | 9.02 | N/A | 8.65 | 19.0 | 19.3 | 18.4 | 19.2 | 19.3 |
| 12 | | | | | | | | | | |
| 2020/MA/0001- | 20 | 1.39:1 | 8.65 | N/A | 8.52 | 18.2 | 19.8 | 19.1 | 20.3 | 19.5 |
| 13 | | | | | | | | | | |
| 2020/MA/0001- | 20 | 1.39:1 | 8.32 | 8.35 | 8.76 | 19.3 | 18.7 | 17.6 | 19.9 | 18.3 |
| 14A | | | | | | | | | | |
| 2020/MA/0001- | 20 | 1.39:1 | 8.84 | 8.33 | 8.74 | 19.3 | 17.8 | 18.1 | 18.4 | 19.5 |
| 14B | | | | | | | | | | |

ALow VLX-1005 content was attributed to undissolved drug due to low NaOH to VLX-1005 ratio and to HPLC diluent concentration; originally 60:40 ACN:Water was adjusted to 75:25 ACN:Water for 100% recovery
^{B}This portion of the 2020/MA/0001-6C batch was subjected to an additional 30 minutes of vortexing and sonication (high energy)

TABLE 10
Compound 1 Solution Stability Evaluations^{c}

| | Storage | Stability | |
|---|---|---|---|
| Batch | Condition | Timepoint (hr.) | Assay(%) |
| 2020/MA/0001-29 | RT | 2.5 | 101.7 |
| | 5° C. | | 101.1 |
| | RT | 4.5 | 98.8 |
| | 5° C. | | 99.9 |
| | RT | 6 | 99.9 |
| | 5° C. | | 101.1 |
| | RT | 12 | 101.1 |
| | 5° C. | | 101.9 |
| | RT | 24 | 98.4 |
| | 5° C | | 100.2 |
| | RT | 36 | 98.2 |
| | 5° C. | | 98.1 |
| | 5° C. | 48 | 99.1 |

^{c}15 mg VLX-1005/mL in 25% HP-β-CD at a 1.6:1NaOH:VLX-1005 molar ratio with final pH of 8.47

### Example 3: Comparative Solubility of Compound 1 in Cyclodextrins at Basic pH

Solubility of Compound 1 was tested in SBE-P-CD at a pH of 9.5 to determine if improvement in solubility at higher pH is specific to HP-β-CD or applies to other cyclodextrins as well.

For this study, 1.20 g of SBE-β-CD was weighed into a beaker and 1.31 mL of water was added. This mixture was stirred to dissolve solid completely. 0.846 g of 60% SBE-β-CD stock solution was weighed into a sample vial, and 1.53 mg of GLY, 70 μL of water, and 130 μL of 2N NaOH were added. This mixture was stirred for 10 minutes at room temperature to dissolve solid completely. 80.6 mg of Compound 1 was added (80 mg/mL) and the mixture was sonicated for 10 minutes followed by vigorous stirring for 22 hours at room temperature (23-25° C.).

Visual observation indicated that no clear solution was obtained. Little, if any, dissolution of Compound 1 was obtained using SBE-P-CD.

### Example 4: Exemplary IV Formulation of Compound 1

Compound 1 Solution (15 mg/mL) for IV Infusion

61.25-63.75 g of hydroxypropyl-β-cyclodextrin (HP-β-CD) is dissolved in 125 mL of sterile water with continuous stirring. Mixing is continued until a clear solution is obtained.

Dilute (e.g., 0.5 N) sodium hydroxide at a target 1.6:1 NaOH:Compound 1 molar ratio is slowly added to the HP-β-CD solution while continuously stirring. The resulting basic pH of about 9.5 to 12 facilitates the dissolution of the active ingredient (Compound 1).

The active ingredient (e.g., 3.675 to 3.825 g) Compound 1) is slowly added under vigorous stirring. Stirring is continued for an additional 10 min followed by 10 min of sonication. Alternate stirring and sonication cycles are repeated at least three (3) times until the active ingredient is dissolved.

Under continuous stirring, dilute hydrochloric acid (0.2 N) is slowly added to a target pH range of 8.4 to 8.7 if necessary.

Water is added to achieve the target concentration of 15 mg of Compound 1 per mL.

The solution is filtered through a 0.22 m filter and stored at 2-8° C. Prior to administration, the solution is sterile filtered under aseptic processing into syringes of appropriate size.

The resulting Compound 1 Solution (15 mg/mL) can be used as-is or further diluted with HP-β-CD solution for IV administration. For example, the Compound 1 Solution (15 mg/mL) can be diluted 1:10 to obtain a 1.5 mg/mL Compound 1 IV formulation.

### Example 5: Formulation of Compound 1 with NaOH Titration

Compound 1 Solution (15 mg/mL) for IV Infusion

12.5 g of hydroxypropyl-O-cyclodextrin (HP-β-CD) is dissolved in 20 mL of sterile water with continuous stirring. Mixing is continued until a clear solution is obtained.

Dilute (e.g., 0.5 N) sodium hydroxide at a target 1.6:1 NaOH:Compound 1 molar ratio is added in two portions; one to the HP-β-CD solution while continuously stirring and a second portion after API addition. The first portion added to the HP-β-CD solution before addition of the active ingredient is 1.9 mL.

The active ingredient (e.g., 746.25 to 753.75 mg Compound 1) is then slowly added under vigorous stirring. Stirring is continued is continued until API is wetted and dispersed. The second portion of NaOH (about 3.1 mL) is added until the pH is increased sufficiently to attain complete API dissolution.

Under continuous stirring, dilute hydrochloric acid (0.2 N) is slowly added to a target pH range of 8.4 to 8.7.

Water is added to achieve the target concentration of 15 mg of Compound 1 per mL.

The solution is filtered through a 0.22 m filter and stored at 2-8° C.

### Example 6: Formulation of Compound 1 without Sonication

Compound 1 Solution (15 mg/mL) for IV Infusion

750 g of hydroxypropyl-β-cyclodextrin (HP-β-CD) is dissolved in 1200 mL of sterile water with continuous stirring. Mixing is continued until a clear solution is obtained.

Dilute (e.g., 0.5 N) sodium hydroxide at a target 1.6:1 NaOH:Compound 1 molar ratio is slowly added to the HP-β-CD solution while continuously stirring. The resulting basic pH of about 9.5 to 12 facilitates the dissolution of the active ingredient (Compound 1).

The active ingredient (e.g., 45.0 g) Compound 1) is slowly added under vigorous stirring. Stirring is continued without sonication for up to one hour until API is dissolved.

Under continuous stirring, dilute hydrochloric acid (0.2 N) is slowly added to a target pH range of 8.4 to 8.7.

Water is added to achieve the target concentration of 15 mg of Compound 1 per mL. The total solution volume is 3 liters.

The solution is filtered through a 0.22 m filter and stored at 2-8° C.

### Example 7: Formulation of Compound 1 with Lower NaOH Ratio

Compound 1 Solution (15 mg/mL) for IV Infusion

62.5 g of hydroxypropyl-O-cyclodextrin (HP-β-CD) is dissolved in 125 mL of sterile water with continuous stirring. Mixing is continued until a clear solution is obtained.

21.2 mL of dilute (e.g., 0.5 N) sodium hydroxide is slowly added to the HP-β-CD solution while continuously stirring. The resulting basic pH of about 9 to 11 facilitates the dissolution of the active ingredient (Compound 1).

The active ingredient (e.g., 3.675 to 3.825 g) Compound 1) is slowly added under vigorous stirring. Stirring is continued for an additional 10 min followed by 10 min of sonication. Alternate stirring and sonication cycles are repeated at least three (3) times until the active ingredient is dissolved.

Under continuous stirring, 27.3 mL of dilute hydrochloric acid (0.2 N) is slowly added resulting in a pH of 6.5-8. Additional NaOH (about 6 mL) is added to attain the target pH range of 8.4 to 8.7.

Water is added to achieve the target concentration of 15 mg of Compound 1 per mL.

The solution is filtered through a 0.22 m filter and stored at 2-8° C.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. An aqueous pharmaceutical formulation comprising: 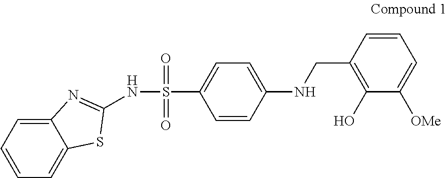 [SMILES: COc1cccc(CNc2ccc(S(=O)(=O)Nc3nc4ccccc4s3)cc2)c1O]
or a pharmaceutically acceptable salt thereof; and
2-hydroxypropyl-β-cyclodextrin (HP-β-CD) in an amount of between about 1% and 50% w/v,
wherein the formulation has a pH of between about 6.0 and 10.

2. An aqueous pharmaceutical formulation comprising: 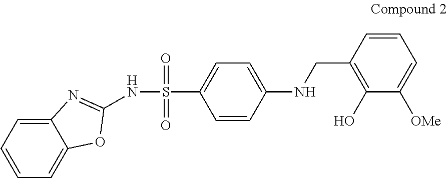 [SMILES: COc1cccc(CNc2ccc(S(=O)(=O)Nc3nc4ccccc4o3)cc2)c1O]
or a pharmaceutically acceptable salt thereof, or diastereomers thereof and
2-hydroxypropyl-β-cyclodextrin (HP-β-CD) in an amount of between about 1% and 500% w/v,
wherein the formulation has a pH of between about 6.0 and 10.

3. An aqueous pharmaceutical formulation comprising: 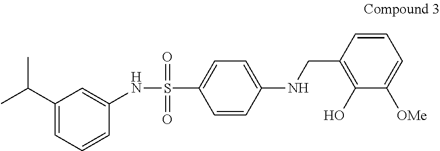 [SMILES: COc1cccc(CNc2ccc(S(=O)(=O)Nc3cccc(C(C)C)c3)cc2)c1O]
or a pharmaceutically acceptable salt thereof, or diastereomers thereof; and
2-hydroxypropyl-β-cyclodextrin (HP-β-CD) in an amount of between about 1% and 50% w/v,
wherein the formulation has a pH of between about 6.0 and 10.

4. The aqueous pharmaceutical formulation of any one of claims 1-3, wherein the 2-hydroxypropyl-β-cyclodextrin (HP-β-CD) is in an amount between about 5%-15%, 10%-20%, 15%-25%, 25%-40%, 35%-45%, or 45%-50% w/v.

5. The aqueous pharmaceutical formulation of claim 4, wherein the 2-hydroxypropyl-β-cyclodextrin (HP-β-CD) is in an amount of about 10%, 15%, 20%, 25%, 30%, 35%, 40%, 45%, or 50% w/v.

6. The aqueous pharmaceutical formulation of any one of claims 1-5, wherein the compound of Formula (I) is in an amount between about 1 mg/mL to about 100 mg/mL.

7. The aqueous pharmaceutical formulation of claim 6, wherein the compound of Formula (I) is in an amount between about 10-90 mg/mL, 50-100 mg/mL, 20-50 mg/mL, or 35-95 mg/mL.

8. The aqueous pharmaceutical formulation of any one of claims 1-7, wherein the composition has a pH in the range of 8.0 and 9.5.

9. The aqueous pharmaceutical formulation of claim 8, wherein the pH is in the range of 8.4 to 8.7.

10. The aqueous pharmaceutical formulation of any one of claims 1-9, wherein the formulation further comprises a pharmaceutically acceptable carrier, additive, excipient, preservative, solvent, buffer, or a mixture thereof.

11. The aqueous pharmaceutical formulation of any one of claims 1-10, wherein the formulation is suitable for parenteral administration.

12. The aqueous pharmaceutical formulation of claim 11, wherein parenteral administration is selected from the group consisting of subcutaneous injection, intravenous injection, intramuscular injection, intraperitoneal injection, infusion techniques, or a combination thereof.

13. The aqueous pharmaceutical formulation of claim 12, wherein the formulation is an intravenous formulation.

14. The aqueous pharmaceutical formulation of any one of claims 1-13 for use as a therapeutic agent in the treatment or prevention of 12-LOX mediated diseases and disorders.

15. A method for treating or preventing a 12-LOX mediated disease and/or disorder comprising administering the aqueous pharmaceutical formulation of any one of claims 1-13 to a subject in need thereof.

16. The method of claim 15, wherein the subject in need thereof is a mammal.

17. The method of claim 15 or 16, wherein the subject in need thereof is a human.

18. The method of any one of claims 15-17, wherein the subject in need thereof has a 12-LOX mediated disease and/or disorder.

19. The method of any one of claims 15-18, wherein the 12-LOX mediated disease and/or disorder is selected from the group consisting of: type 1 diabetes, type 2 diabetes, diabetic kidney disease, diabetic nerve disease, cardiovascular disease, Alzheimer's disease, Non-Alcoholic steatohepatitis, platelet hemostasis, skin diseases, heparin-induced thrombocytopenia, thrombosis, lupus, and cancer.

20. The method of any one of claims 16-19, wherein the aqueous pharmaceutical formulation is administered by infusion.

21. The method of claim 20, wherein the infusion comprises infusing between about 1 and 1,000 mg of compound 1 over 30 minutes to 24 hours.

22. The method of claim 21, wherein the infusion is for about 1, 2, 3, 4, or 5 hours.

23. The method of claim 22, wherein the amount of the compound 1 infused is about 10 to 1,000 mg.

24. The method of any one of claims 21-23, wherein the infusion is repeated over between about 1 and 14 days.

25. The method of claim 24, wherein the infusion is repeated over 14 days.

26. The method of any one of claims 15-19, wherein the administration is by a bolus.

27. A method of making the pharmaceutical formulation of any of one claims 1 to 13, comprising:
(a) dissolving hydroxypropyl-β-cyclodextrin (HP-β-CD) in water to obtain a clear HP-β-CD solution;
(b) adding sodium hydroxide to the HP-β-CD solution while stirring to obtain a basic HP-β-CD solution of between about 9.5 and 12;
(c) adding the compound of Formula (I) slowly under agitation;
(d) optionally adding additional sodium hydroxide while stirring;
(e) stirring for at least 10 minutes optionally followed by at least 10 minutes of sonication at least three times until the compound of Formula (I) is dissolved; and
(f) adding hydrochloric acid while stirring to result in a solution with a pH in a range from 8.4 to 8.7
(g) q.s. to final volume with water.
